(19) United States
(12) Patent Application Publication
(10) Pub. No.: US 2026/0175206 A1
(43) Pub. Date: Jun. 25, 2026

(54) CATALYTIC METHOD FOR SELECTIVE CYCLO-AMINATION OF ALKANOLAMINE AND DIOL TO PRODUCE ON-PURPOSE CYCLIC ETHYLENEAMINES OF PIPERAZINE AND DERIVATIVES

(71) Applicant: UOP LLC, Des Plaines, IL (US)

(72) Inventors: Nabil Al Yassir, Ontario (CA); Carl J. Stevens, Seaside, CA (US); Edwin P. Boldingh, Arlington Heights, IL (US)

(21) Appl. No.: 19/404,267

(22) Filed: Dec. 1, 2025

Related U.S. Application Data
(60) Provisional application No. 63/737,268, filed on Dec. 20, 2024.

(51) Int. Cl. B01J 29/70; C07D 295/027; C07D 487/08

(52) U.S. Cl. CPC B01J 29/7049; C07D 295/027; C07D 487/08

(57) ABSTRACT

Catalytic processes to produce on purpose piperazine are described. The processes are based on the use of a 10-member ring zeolite, preferably with MFI topology, with tunned morphology and acidity to promote intermolecular or intramolecular cyclization of alkanolamines, ethyleneamines, or diols to cyclic piperazine. The unavoidable byproducts due to thermodynamics can be tuned to favor piperazine upon recycling. The processes can be used to manufacture piperazine which can be used as solvent for post-combustion CO₂ capture.

### RELATED APPLICATIONS

This application claims priority to U.S. Provisional Patent Application Ser. No. 63/737,268, filed on Dec. 20, 2024, the entirety of which is incorporated herein by reference.

### BACKGROUND

Cyclic diamine piperazine is one of the leading solvent candidates for CO₂ capture processes from post-combustion sources, such as flue gas, offered by multiple technology providers. Many other solvents are not suitable because they have lower CO₂ capacity and high levels of thermal and oxidative degradation that prevent operation at high temperatures.

Currently, piperazine is only made as a by-product of ethylene amines commercial technologies (viz. reductive amination of ethylene dichloride (EDC) or monoethanolamine (MEA)). These processes have limited yield (e.g., a maximum of 10% of the total products) and limited market supply participants.

US Application No. 2004/0371452 describes a method for the reductive amination of diethanolamine to form a product composition that includes piperazine and aminoethylethanolamine along with other by-products. A catalyst with a transitional alumina/second metal oxide support and a mixture of catalytic metal is used for the reaction. As shown in Examples 1-3 and Tables 2-4, the combined amount of piperazine and aminoethylethanolamine in the product composition is 75 wt % or greater of the products. The piperazine in the product composition is 10-40 wt % of the products.

The market growth of ethylene amines is about 3.7% compound annual growth rate (CAGR) whereas piperazine growth is about 10-20% CAGR. It is expected that piperazine capacity will be insufficient to meet the demand if the market adopts piperazine for carbon capture processes.

Therefore, there is a need for processes to produce piperazine having increased yield and decreased byproducts.

### DESCRIPTION

The present invention provides catalytic processes to produce on purpose piperazine. The processes are based on the use of a 10-member ring zeolite, preferably with MFI topology, with tuned morphology and acidity to promote intermolecular or intramolecular cyclization of alkanolamines, ethyleneamines, or diols, to cyclic piperazine. The unavoidable byproducts due to thermodynamics can be tuned to favor piperazine upon recycling. Leading catalytic prototypes will be used to manufacture piperazine which will be used as solvent for a variety of processes, including post-combustion CO₂ capture through advanced solvent carbon capture (ASCC) technology, sulfur extraction from liquified petroleum gas (LPG) streams, and the like.

One aspect of the invention is a process for the production of piperazine. In one embodiment, the process comprises reacting an alkanolamine or an ethyleneamine or a diol in a reaction zone comprising a reactor in the presence of anhydrous ammonia and a catalyst comprising a 10-member ring zeolite to produce a reaction mixture comprising piperazine.

The anhydrous ammonia is injected along with the feed to convert the hydroxyl groups to NH₂ groups.

In some embodiments, the reaction conditions include a temperature in the range of 285° C. to 350° C., and a pressure in the range of 1700-2500 psig, or both.

In some embodiments, the reaction mixture further comprises a piperazine derivative and the process further comprises: separating the reaction mixture into a piperazine stream comprising the piperazine and a byproduct stream comprising the piperazine derivative; and recycling the byproduct stream to the reaction zone.

In some embodiments, the piperazine derivative comprises at least one of an alkylated piperazine, such as N-methylpiperazine including N,N-Dimethylpiperazine, triethylenediamine (TEDA), aminoethylpiperazine (AEP), and hydroxyethylpiperazine (HEP).

The characteristics needed for the alkanolamine, ethyleneamine, or diol are reactivity and the level of NH₂ containing groups. Raw material cost is also a factor in the selection.

Any suitable alkanolamine can be used. Suitable alkanolamines include, but are not limited to, diethanolamine, or monoethanolamine, or triethanolamine, or combinations thereof.

Any suitable ethyleneamine can be used. Suitable amines include, but are not limited to, ethylenediamine.

Any suitable diol can be used. Suitable diols include, but are not limited to, ethylene glycol. The catalyst comprises a 10-member ring zeolite. In some embodiments, the 10-member ring zeolite comprises an MFI-type zeolite. Suitable 10-member ring zeolites include, but are not limited to, ZSM-5, MEL zeolites, MTT zeolites, TUN zeolites, and MWW zeolites.

In some embodiments, the 10-member ring zeolite is modified with phosphorus. The phosphorus may comprise phosphoric acid, phosphorous oxide, ammonium dihydrogen phosphate, phosphomolybdic acid, or combinations thereof.

In some embodiments, the 10-member ring zeolite is modified with gallium.

In some embodiments, the 10-member ring zeolite is modified by silylation. The external surface of the zeolite is passivated through the chemical liquid deposition of silica. The silica deposition can be repeated one or more times, typically from 1 to 6 depositions.

In some embodiments, the catalyst has a mole ratio of Si to Al in a range of 10-140.

In some embodiments, the catalyst has an acidity in a range of 0.75 to 1.20 mmol/g.

In some embodiments, the catalyst has a mole ratio of Si to Al in a range of 10-140 and has an acidity in a range of 0.75 to 1.20 mmol/g.

In some embodiments, the alkanolamine comprises diethanolamine and the mole ratio of anhydrous ammonia to diethanolamine is in a range of 10 to 100.

Another aspect of the invention is a process for the production of piperazine. In one embodiments, the process comprises: reacting an alkanolamine or ethylenediamine or diol in a reaction zone comprising a reactor in the presence of anhydrous ammonia and a catalyst comprising a 10-member ring zeolite to produce a reaction mixture comprising piperazine and a piperazine derivative; separating the reaction mixture into a piperazine stream comprising the piperazine and a byproduct stream comprising the piperazine derivative; and recycling the byproduct stream to the reaction zone.

In some embodiments, the piperazine derivative comprises at least one of an alkylated piperazine, such as N-methylpiperazine including N,N-Dimethylpiperazine, triethylenediamine (TEDA (DABCO)), aminoethylpiperazine (AEP), and hydroxyethylpiperazine (HEP).

Any suitable alkanolamine can be used. Suitable alkanolamines include, but are not limited to, diethanolamine, or monoethanolamine, or triethanolamine, or combinations thereof.

Any suitable diol can be used. Suitable diols include, but are not limited to, ethylene glycol.

The catalyst comprises a 10-member ring zeolite. In some embodiments, the 10-member ring zeolite comprises an MFI-type zeolite.

In some embodiments, the 10-member ring zeolite is modified with phosphorus. The phosphorus may comprise phosphoric acid, phosphorous oxide, ammonium dihydrogen phosphate, phosphomolybdic acid, or combinations thereof.

In some embodiments, the 10-member ring zeolite is modified by silylation. The external surface of the zeolite is passivated through the chemical liquid deposition of silica. The silica deposition can be repeated one or more times, typically from 1 to 6 depositions.

In some embodiments, the catalyst has a mole ratio of Si to Al in a range of 10-140.

In some embodiments, the catalyst has an acidity in a range of 0.75 to 1.20 mmol/g.

In some embodiments, the alkanolamine comprises diethanolamine and the mole ratio of anhydrous ammonia to diethanolamine is in a range of 10 to 100.

### EXAMPLES

The following examples illustrate aspects of the invention.

### Example 1

Selective cyclo-amination of diethanolamine (DEA) was conducted in a high pressure stirred SS316 batch reactor. The catalyst, which is added in powdered form, was activated ex.situ at 275° C. for 10 hours under nitrogen flow. Examples of catalysts include MFI (ZSM-5) zeolite of variable SiO₂/Al₂O₃ ratio (23-280) (MFI23, 23 represents ZSM-5 with SiO₂/Al₂O₃), small (50-100 nm) and large (2 micron) ZSM-5 crystals, FAU zeolite (HY5, USY5-30), amorphous silica-alumina (ASA, SiO₂/Al₂O₃=2), metal-containing MFI (Gallium (Ga), Iron (Fe)), Phosphorous (P) modified MFI23, and silylated (SiO₂ passivated) MFI. A certain amount of feed mixture and liquified anhydrous ammonia were charged into the vessel, with a molar ratio of NH₃ to feed of 10-100. The mixture was heated (280-350° C.) under stirring for 30 min. to 10 hours on stream under autogenic pressure (1700-2500 psig). Liquid products were analyzed offline using gas chromatography equipped with flame ionization detector (FID) and CP-Volamine 60 m×0.32 mm i.d×5 μm.

The gas chromatography analysis of the products from DEA selective animation is shown in Table 1. This example shows that piperazine can be prepared in good yield from DEA animation with careful selection of catalyst and process parameters. As can be seen from Table 1, there was almost full conversion of diethanolamine to piperazine and derivatives at the given conditions. Selectivity to piperazine (17-52.1 wt. %) varies with catalyst structure and composition in which a zeolite of MFI topology (ZSM-5) and high acidity exhibits the highest selectivity. The selectivity to piperazine is reduced to bulky piperazine derivatives (AEP, HEP) over a large pore zeolite such as 12-MR FAU. A linear relationship of PZ selectivity is obtained with increasing NH₃/DEA molar ratio up to 90, whereas at ratios greater than 90, a slight drop can be seen. The highest PZ selectivity is about 52.1%.

TABLE 1
PZ synthesis through selective cyclo-amination of diethanolamine at 300°
C., 4 HOS, NH₃/DEA (molar) = 90, 145-155 barg (autogenic NH₃)

| | | Product Selectivity (%) | | | | | | | | |
|---|---|---|---|---|---|---|---|---|---|---|
| Catalyst/ | X_{DEA} | | 1- | | | AEP + | | | | |
| Parameter | % | PZ | MPZ | EDA | TEDA | HEP | EPZ | DETA | MEA | unk |
| MFI23 | 100 | 52.1 | 4.35 | 18.6 | 9.18 | 13.8 | 1.89 | — | — | — |
| MFI23 - | 100 | 48.0 | 2.28 | 18.0 | 7.91 | 10.4 | 3.9 | 2.75 | — | 6.75 |
| 10 HOS | | | | | | | | | | |
| MFI80 - | 95 | 22.1 | — | 18.9 | 16.1 | 23.1 | — | — | 19.8 | — |
| 1 HOS | | | | | | | | | | |
| MFI80 - | 100 | 46.4 | 11.7 | 16.9 | 9.51 | 15.5 | — | — | — | — |
| 10 HOS | | | | | | | | | | |
| MFI30 - | 100 | 42.1 | 21.8 | 14.5 | 7.66 | 13.8 | — | — | — | — |
| 10 HOS | | | | | | | | | | |
| 2% Fe/MFI30 - | 100 | 43.6 | 9.22 | 17.5 | 7.06 | 10.5 | 3.11 | 2.05 | — | 7.00 |
| 10 HOS | | | | | | | | | | |
| MFI30L^{a} | 100 | 42.5 | 15.7 | 14.7 | 9.90 | 15.2 | — | — | 2.0 | — |
| MFI30N^{b} | 100 | 46.7 | 16.2 | 14.1 | 8.1 | 13.5 | — | — | 1.2 | — |
| 2% Ga/MFI40 | 100 | 23.1 | 14.1 | 23.9 | 9.20 | 28.9 | — | — | — | 0.80 |
| UZM54 - | 100 | 38.6 | 26.4 | 13.9 | 6.75 | 12.8 | 1.59 | — | — | — |
| 10 HOS | | | | | | | | | | |
| ZSM23 - | 97 | 20.8 | — | 28.8 | 9.27 | 23.1 | — | 3.83 | 14.2 | — |
| 10 HOS | | | | | | | | | | |
| HY5 - | 100 | 17 | 25.1 | 20.6 | — | 37.3 | — | — | — | — |
| 8 HOS (99) | | | | | | | — | — | — | — |
| USY5 - | 91 | 19.1 | 20.7 | 24.9 | — | 33.1 | — | — | — | 2.2 |
| 10 HOS | | | | | | | | | | |
| ASA | 100 | 20.9 | 5.31 | 19.9 | 14.6 | 30.82 | — | 2.49 | 5.99 | — |

^{a}large crystals;
^{b}nanocrystals;
Number in parentheses represents NH₃/DEA molar ratio;
unk: Unknown.

### Example 2

Data for selective cyclo-amination of monoethanolamine (MEA) (Example 1 procedures) are shown in Table 2. Similar to DEA animation, piperazine was obtained in high yield over highly acidic ZSM-5. Higher PZ yield was observed with increasing NH₃/MEA molar ratios (similar to DEA animation) and over acidic large ZSM-5 crystals.

TABLE 2
PZ synthesis through selective cyclo-amination of monoethanolamine (MEA)
at 300° C., 4 HOS, NH₃/MEA (molar) = 15-100, 145-155 barg (autogenic NH₃)

| | | Product Selectivity (%) | | | | | | | |
|---|---|---|---|---|---|---|---|---|---|
| Catalyst/ | X_{MEA} | | 1- | | | AEP + | | | |
| Parameter | % | PZ | MPZ | EDA | TEDA | HEP | EPZ | DETA | ukn |
| MFI23 (17) | 100 | 43.5 | — | 17.9 | 13.3 | 20.2 | 2.60 | 2.55 | — |
| MFI23 (34) | 96 | 48.7 | — | 19.1 | 12.3 | 19.9 | — | — | — |
| MFI23 (53) | 100 | 52.1 | 4.88 | 20.1 | 9.42 | 13.5 | — | — | — |
| MFI23 (96) | 100 | 53.1 | 5.10 | 26.2 | 6.03 | 9.60 | — | — | — |
| MFI23 - | 100 | 54.1 | 12.3 | 18.4 | 2.15 | 11.3 | — | — | 1.75 |
| 10 HOS (57) | | | | | | | | | |
| MFI23 - | 100 | 44.6 | 18.9 | 17.6 | 3.73 | 10.2 | — | — | 4.97 |
| 15 HOS (53) | | | | | | | | | |
| MFI30L^{a} - | 45 | 37.1 | — | 38.4 | 8.97 | 15.6 | — | — | — |
| 0.5 HOS (53) | | | | | | | | | |
| MFI30L - | 72 | 45.6 | — | 28.8 | 8.90 | 16.7 | — | — | — |
| 2 HOS (50) | | | | | | | | | |
| MFI30L - | 100 | 56.5 | 4.77 | 16.9 | 8.63 | 13.2 | — | — | — |
| 10 HOS (53) | | | | | | | | | |
| MFI30N^{b} | 100 | 51.6 | 12.8 | 14.9 | 8.10 | 12.7 | — | — | — |
| 10 HOS (50) | | | | | | | | | |
| 2% Fe/MFI30 | 91 | 45.4 | 7.11 | 23.7 | 6.88 | 14.6 | — | — | 2.31 |
| (51) | | | | | | | | | |

^{a}large crystals;
^{b}nanocrystals;
Number in parentheses represents NH3/MEA molar ratio;
unk: Unknown

### Example 3

Additional feed having lower intrinsic reactivity was explored for piperazine synthesis, ethylene glycol (EG), following the procedures of example 1. Piperazine was synthesized from cost-effective ethylene glycol with lower yield compared to DEA and MEA, despite comparable selectivity. Piperazine yield slightly increased with increasing NH₃/EG ratio. Aggressive process parameters (higher temperature, and longer hours on stream) resulted in higher PZ yield. 2- to 4-fold increase in PZ yield was obtained over phosphorous modified MFI23.

TABLE 3
PZ synthesis through selective cyclo-amination of ethylene glycol (EG)
at 300° C., 4 HOS, NH₃/EG (molar) = 15-100, 145-155 barg (autogenic NH₃)

| | | Product Selectivity (%) | | | | | | | | |
|---|---|---|---|---|---|---|---|---|---|---|
| Catalyst/ | X_{EG} | | 1- | | | AEP + | | | | |
| Parameter | % | PZ | MPZ | EDA | TEDA | HEP | EPZ | DETA | MEA | ukn |
| MFI23 (16) | 27.3 | 44.9 | — | 21.6 | 10.1 | 16.3 | — | — | 7.10 | — |
| MFI23 (32) | 26.2 | 43.7 | — | 32.2 | 5.92 | 8.86 | — | — | 9.34 | — |
| MFI23 (54) | 28.1 | 45.3 | — | 41.6 | — | 3.21 | — | — | 9.88 | — |
| MFI23 (90) | 31.7 | 43.3 | — | 42.7 | — | 5.25 | — | — | 8.40 | 0.35 |
| MFI23 (17) | 39.3 | 48.3 | — | 19.5 | 15.1 | 17.1 | — | — | — | — |
| 10 HOS | | | | | | | | | | |
| MFI23 (17) | 33.4 | 44.1 | — | 18.6 | 19.8 | 17.5 | — | — | — | — |
| 20 HOS | | | | | | | | | | |
| 4% PMIF23 (26) | 90.9 | 31.5 | — | 22.9 | 11.6 | 23.1 | — | — | 4.21 | 6.69 |
| 7% PMFI23 (25) | 96.6 | 19.4 | — | 28.6 | 11.4 | 29.9 | — | — | 3.13 | 7.59 |
| 4% PMFI23 (52) | 87.2 | 44.3 | 12.6 | 22.4 | 7.81 | 12.9 | — | — | — | — |
| 7% PMFI23 (52) | 100 | 32.3 | — | 40.9 | 9.71 | 17.1 | — | — | — | — |
| 4% PMFI23 (99) | 71.8 | 49.3 | 5.60 | 31.3 | 5.06 | 8.75 | — | — | — | — |
| 7% PMFI23 (95) | 100 | 51.4 | — | 32.5 | 7.00 | 9.10 | — | — | — | — |
| HY5 | 19.2 | 21.6 | — | 44.3 | — | 18.6 | — | — | 12.5 | 3.0 |

Number in parentheses represents NH₃/EG molar ratio;
unk: Unknown

### Example 4

Table 4 shows that selective cyclo-(autogenic amination) of EDA resulted in high selectivity to piperazine compared to EG, MEA, and DEA. Upon co-feeding anhydrous ammonia (NH₃/EDA=6-25), a 1.5-fold increase in piperazine yield was noted. Passivation of MFI zeolite with TEOS (silylation) increased the activity to piperazine synthesis. The activity of SiO₂ passivated MFI zeolite is shown to be dependent on zeolite crystal size and starting MFI (pristine vs. NH₄ form). PZ selectivity increased from 54.1% to 64.4% for silylated NH₄MFI30L (20% increase in conversion).

TABLE 4
PZ synthesis through selective cyclization ethylenediamine
(EDA) at 300° C., 1 HOS, 145-155 barg (autogenic)

| | | Product Selectivity (%) | | | | | | | |
|---|---|---|---|---|---|---|---|---|---|
| Catalyst/ | X_{EDA} | | 1- | | AEP + | | | | |
| Parameter | % | PZ | MPZ | TEDA | HEP | EPZ | DETA | MEA | ukn |
| MFI23 | 54.2 | 51.9 | — | 23.1 | 21.8 | — | 3.20 | — | — |
| MFI30L^{a} | 54.1 | 55.7 | — | 14.8 | 20.8 | — | 8.70 | — | — |
| 2% Ga/MFI40 | 44.4 | 61.9 | — | 5.41 | 24.1 | — | 8.59 | — | — |
| MFI30L (6) | 71.6 | 57.1 | — | 10.2 | 28.8 | — | 3.93 | — | — |
| MFI30L (12) | 71.3 | 59.4 | — | 7.31 | 29.6 | — | 3.78 | — | — |
| 2% Ga/ | 55.6 | 67.5 | — | — | 24.1 | — | 8.40 | — | — |
| MFI40 (6) | | | | | | | | | |
| 2% Ga/ | 58.7 | 61.0 | — | 2.56 | 30.2 | — | 6.24 | — | — |
| MFI40 (9) | | | | | | | | | |
| 2% Ga/ | 63.7 | 61.7 | — | 3.13 | 30.3 | — | 4.91 | — | — |
| MFI40 (25) | | | | | | | | | |
| MFI30L | 17.1 | 54.1 | — | — | 13.4 | — | 32.5 | — | — |
| (n = 1, 6% | | | | | | | | | |
| SiO₂) | | | | | | | | | |
| NH4MFI30L | 57.5 | 58.9 | — | 13.3 | 22.5 | — | 5.30 | — | — |
| (n = 1, 6% | | | | | | | | | |
| SiO₂) | | | | | | | | | |
| NH4MFI30L | 64.2 | 64.4 | — | 10.3 | 21.4 | — | 3.95 | — | — |
| (n = 1, 6% | | | | | | | | | |
| SiO₂) - 4 HOS | | | | | | | | | |

^{a}large crystals;
Number in parentheses represents NH₃/EDA molar ratio;
unk: Unknown

### Example 5

Recycling experiments of piperazine derivatives were conducted following the procedure of Example 4. The recycling feeds are simulated based on EDA selective cyclo-amination over 2% Ga/MFI40 (NH₃/EDA=25) (Table 4). Full recycle (feed+product) corresponds to unconverted EDA and piperazine derivatives (TEDA, AEP, DETA) products. The other recycle feed is piperazine derivatives product (TEDA, AEP, DETA) (PZ derivative recycle). Example 5 illustrates that on-purpose piperazine is achievable upon recycling piperazine derivative products with EDA. As can be seen from Table 5, full recycle is converted to piperazine with 80% selectivity and 45% EDA conversion. Higher PZ yield is seen upon cyclo-amination using anhydrous NH₃ (NH₃/EDA=35). Recycling unconverted EDA (and potentially fresh EDA) facilitates the conversion of piperazine derivatives to piperazine.

TABLE 5
Recycling of piperazine derivatives at 300°
C., 1 HOS, 150 barg (autogenic) over 2% Ga/MFI40

| | | | Sel (%) | | | | |
|---|---|---|---|---|---|---|---|
| | NH3/EDA | Conversion, | | | | AEP + | |
| Feed | (molar) | % | PZ | EDA | TEDA | HEP | DETA |
| Full Recycle | — | 45.0 | 80.3 | — | 19.7 | — | — |
| (Feed + | | | | | | | |
| Product) | | | | | | | |
| Full Recycle | 35 | 73.7 | 87.9 | — | 12.1 | — | — |
| (Feed + | | | | | | | |
| Product) | | | | | | | |
| PZ derivative | 30^{a} | 34.9 | 33.5 | 22.9 | 43.6 | — | — |
| Recycle | | | | | | | |

^{a}NH₃/PZ derivatives (TEDA + AEP + DETA)

### Specific Embodiments

While the following is described in conjunction with specific embodiments, it will be understood that this description is intended to illustrate and not limit the scope of the preceding description and the appended claims.

A first embodiment of the invention is a process for the production of piperazine comprising reacting an alkanolamine or an ethyleneamine or a diol in a reaction zone comprising a reactor in the presence of anhydrous ammonia and a catalyst comprising a 10-member ring zeolite to produce a reaction mixture comprising piperazine. An embodiment of the invention is one, any or all of prior embodiments in this paragraph up through the first embodiment in this paragraph wherein the alkanolamine comprises diethanolamine, or monoethanolamine, or triethanolamine, or combinations thereof An embodiment of the invention is one, any or all of prior embodiments in this paragraph up through the first embodiment in this paragraph wherein the diol comprises ethylene glycol. An embodiment of the invention is one, any or all of prior embodiments in this paragraph up through the first embodiment in this paragraph wherein the ethyleneamine comprises ethylenediamine. An embodiment of the invention is one, any or all of prior embodiments in this paragraph up through the first embodiment in this paragraph wherein the 10-member ring zeolite comprises an MFI-type zeolite. An embodiment of the invention is one, any or all of prior embodiments in this paragraph up through the first embodiment in this paragraph wherein the 10-member ring zeolite is modified with phosphorus. An embodiment of the invention is one, any or all of prior embodiments in this paragraph up through the first embodiment in this paragraph wherein the phosphorus comprises phosphoric acid, phosphorous oxide, ammonium dihydrogen phosphate, phosphomolybdic acid, or combinations thereof. An embodiment of the invention is one, any or all of prior embodiments in this paragraph up through the first embodiment in this paragraph wherein the 10-member ring zeolite is modified by silylation. An embodiment of the invention is one, any or all of prior embodiments in this paragraph up through the first embodiment in this paragraph wherein the 10-member ring zeolite is modified by gallium. An embodiment of the invention is one, any or all of prior embodiments in this paragraph up through the first embodiment in this paragraph wherein the catalyst has an acidity in a range of 0.75 to 1.20 mmol/g. An embodiment of the invention is one, any or all of prior embodiments in this paragraph up through the first embodiment in this paragraph wherein the catalyst has a mole ratio of Si to Al in a range of 10-140. An embodiment of the invention is one, any or all of prior embodiments in this paragraph up through the first embodiment in this paragraph wherein the catalyst has a mole ratio of Si to Al in a range of 10-140 and has an acidity in a range of 0.75 to 1.20 mmol/g. An embodiment of the invention is one, any or all of prior embodiments in this paragraph up through the first embodiment in this paragraph wherein the reaction takes place at a temperature in a range of 300° C. to 350° C., and a pressure in a range of 1700-2500 psig. An embodiment of the invention is one, any or all of prior embodiments in this paragraph up through the first embodiment in this paragraph wherein the alkanolamine comprises diethanolamine and wherein a mole ratio of anhydrous ammonia to diethanolamine is in a range of 10 to 100. An embodiment of the invention is one, any or all of prior embodiments in this paragraph up through the first embodiment in this paragraph wherein the reaction mixture further comprises a piperazine derivative and further comprising separating the reaction mixture into a piperazine stream comprising the piperazine and a byproduct stream comprising the piperazine derivative; and recycling the byproduct stream to the reaction zone. An embodiment of the invention is one, any or all of prior embodiments in this paragraph up through the first embodiment in this paragraph wherein the piperazine derivative comprises at least one of an alkylated piperazine, triethylenediamine, aminoethylpiperazine, and hydroxyethylpiperazine.

A second embodiment of the invention is a process for the production of piperazine comprising reacting an alkanolamine or an ethyleneamine or a diol in a reaction zone comprising a reactor in the presence of anhydrous ammonia and a catalyst comprising a 10-member ring zeolite to produce a reaction mixture comprising piperazine and a piperazine derivative; separating the reaction mixture into a piperazine stream comprising the piperazine and a byproduct stream comprising the piperazine derivative; and recycling the byproduct stream to the reaction zone; wherein the reaction takes place at a temperature in a range of 285° C. to 350° C., and a pressure in a range of 1700-2500 psig. An embodiment of the invention is one, any or all of prior embodiments in this paragraph up through the second embodiment in this paragraph wherein the alkanolamine comprises diethanolamine, or monoethanolamine, or triethanolamine, or combinations thereof, or wherein the ethyleneamine comprises ethylenediamine; wherein the diol comprises ethylene glycol. An embodiment of the invention is one, any or all of prior embodiments in this paragraph up through the second embodiment in this paragraph wherein the 10-member ring zeolite comprises an MFI-type zeolite. An embodiment of the invention is one, any or all of prior embodiments in this paragraph up through the second embodiment in this paragraph wherein the 10-member ring zeolite is modified with phosphorus, or is modified with gallium, or is modified by silylation. An embodiment of the invention is one, any or all of prior embodiments in this paragraph up through the second embodiment in this paragraph wherein the catalyst has a mole ratio of Si to Al in a range of 10-140. An embodiment of the invention is one, any or all of prior embodiments in this paragraph up through the second embodiment in this paragraph wherein the catalyst has an acidity in a range of 0.75 to 1.20 mmol/g. An embodiment of the invention is one, any or all of prior embodiments in this paragraph up through the second embodiment in this paragraph wherein the alkanolamine comprises diethanolamine and wherein a mole ratio of anhydrous ammonia to diethanolamine is in a range of 10 to 100.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process for the production of piperazine comprising:
reacting an alkanolamine or an ethyleneamine or a diol in a reaction zone comprising a reactor in the presence of anhydrous ammonia and a catalyst comprising a 10-member ring zeolite to produce a reaction mixture comprising piperazine.

2. The process of claim 1 wherein the alkanolamine comprises diethanolamine, or monoethanolamine, or triethanolamine, or combinations thereof.

3. The process of claim 1 wherein the diol comprises ethylene glycol.

4. The process of claim 1 wherein the ethyleneamine comprises ethylenediamine.

5. The process of claim 1 wherein the 10-member ring zeolite comprises an MFI-type zeolite.

6. The process of claim 1 wherein the 10-member ring zeolite is modified with phosphorus.

7. The process of claim 6 wherein the phosphorus comprises phosphoric acid, phosphorous oxide, ammonium dihydrogen phosphate, phosphomolybdic acid, or combinations thereof.

8. The process of claim 1 wherein the 10-member ring zeolite is modified by silylation.

9. The process of claim 1 wherein the 10-member ring zeolite is modified with gallium.

10. The process of claim 1 wherein the catalyst has a mole ratio of Si to Al in a range of 10-140, and has an acidity in a range of 0.75 to 1.20 mmol/g.

11. The process of claim 1 wherein the reaction takes place at a temperature in a range of 285° C. to 350° C., and pressure in a range of 1700-2500 psig.

12. The process of claim 1 wherein the alkanolamine comprises diethanolamine and wherein a mole ratio of anhydrous ammonia to diethanolamine is in a range of 10 to 100.

13. The process of claim 1 wherein the reaction mixture further comprises a piperazine derivative and further comprising:
separating the reaction mixture into a piperazine stream comprising the piperazine and a byproduct stream comprising the piperazine derivative; and
recycling the byproduct stream to the reaction zone.

14. The process of claim 13 wherein the piperazine derivative comprises at least one of an alkylated piperazine, triethylenediamine, aminoethylpiperazine, and hydroxyethylpiperazine.

15. A process for the production of piperazine comprising:
reacting an alkanolamine or an ethyleneamine or a diol in a reaction zone comprising a reactor in the presence of anhydrous ammonia and a catalyst comprising a 10-member ring zeolite to produce a reaction mixture comprising piperazine and a piperazine derivative;
separating the reaction mixture into a piperazine stream comprising the piperazine and a byproduct stream comprising the piperazine derivative; and
recycling the byproduct stream to the reaction zone; wherein the reaction takes place at a temperature in a range of 285° C. to 350° C., and pressure in a range of 1700-2500 psig.

16. The process of claim 15 wherein the alkanolamine comprises diethanolamine, or monoethanolamine, or triethanolamine, or combinations thereof; or
wherein the ethyleneamine comprises ethylenediamine;
wherein the diol comprises ethylene glycol.

17. The process of claim 15 wherein the 10-member ring zeolite comprises an MFI-type zeolite has a mole ratio of Si/Al in a range of 10-140.

18. The process of claim 15 wherein the 10-member ring zeolite is modified with phosphorus, or is modified with gallium, or is modified by silylation.

19. The process of claim 15 wherein the catalyst has an acidity in a range of 0.75 to 1.20 mmol/g.

20. The process of claim 15 wherein the alkanolamine comprises diethanolamine and wherein a mole ratio of anhydrous ammonia to diethanolamine is in a range of 10 to 100.
